SEQUENCE LISTING (11 sequences)

SEQ ID NO: 1 - DNA, 84 bp, Escherichia coli
  CDS (1)..(84): Helix 1 of E.coli repressor of primer (rop)
accaaacaagaaaaaaccgcccttaacatggcccgctttatcagaagccagacattaacgcttctggagaaactcaacgagctg

SEQ ID NO: 2 - protein, 28 aa, Escherichia coli
TKQEKTALNMARFIRSQTLTLLEKLNEL

SEQ ID NO: 3 - DNA, 84 bp, Escherichia coli
  CDS (1)..(84): Helix 2 of rop
gatgaacaggcagacatctgtgaatcgcttcacgaccacgctgatgagctttaccgcagctgccttgcccgtttcggcgacgac

SEQ ID NO: 4 - protein, 28 aa, Escherichia coli
DEQADICESLHDHADELYRSCLARFGDD

SEQ ID NO: 5 - DNA, 192 bp, Escherichia coli
  CDS (1)..(192): wild type dimeric rop
atgggtaccaaacaagaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgccttgcccgtttcggcgacgacggtgaaaacctg

SEQ ID NO: 6 - protein, 64 aa, Escherichia coli
MGTKQEKTALNMARFIRSQTLTLLEKLNELDADEQADICESLHDHADELYRSCLARFGDDGENL

SEQ ID NO: 7 - DNA, 384 bp, Artificial Sequence
  Description of Artificial Sequence Monomeric rop containing all 4 helices in one continuous sequence
atgggtaccaaacaagaaaaaaccgcccttaacatggcccgctttatcagaagccagacattaacgcttctggagaaactcaacgagctgggtggcggtggcggtaccaaacaagagaagaccgcccttaacatggcccgctttatcagatctcagacattaacgcttctagagaagcttaacgagctcggggcggatgaacaggcagacatatgtgaatcgcttcacgaccacgctgatgagctttaccgcagctgccttgcccgtttcggtggcggtggcggtgcggatgaacaggcagacatctgtgaatcgcttcacgaccacgctgatgagctttaccgcagctgccttgcccgtttcggcgacgacggtgaaaacctg

SEQ ID NO: 8 - protein, 128 aa, Artificial Sequence
  Description of Artificial Sequence Monomeric rop containing all 4 helices in one continuous sequence
MGTKQEKTALNMARFIRSQTLTLLEKLNELGGGGGTKQEKTALNMARFIRSQTLTLLEKLNELGADEQADICESLHDHADELYRSCLARFGGGGGADEQADICESLHDHADELYRSCLARFGDDGENL

SEQ ID NO: 9 - DNA, 19 bp, Artificial Sequence
  Description of Artificial Sequence psp7 upstream amplification sequence
gcgaaattaatacgactca

SEQ ID NO: 10 - DNA, 23 bp, Artificial Sequence
  Description of Artificial Sequence  asp4 downstream amplification sequence
gttggctgctgccaccgctgagc

SEQ ID NO: 11 - protein, 128 aa, Artificial Sequence
  Description of Artificial Sequence RDM14.5
MGTKQEKTALNMARFIRSQTLTLLEKLNELGGGGGTKQEKTALNMARFIRSQTLTHLEKLNELGADEQADICESLADWADELYRSCLARFGGGGGADEQADICESLADWADEHYRSCLARFGDDGENL

(19) United States
(12) Patent Application Publication
(10) Pub. No.: US 2006/0148026 A1
(43) Pub. Date: Jul. 6, 2006

(54) Engineering redox proteins

(75) Inventor: Gianfranco Gilardi, London (GB)

Correspondence Address: SUGHRUE MION, PLLC, 2100 PENNSYLVANIA AVENUE, N.W., SUITE 800, WASHINGTON, DC, 20037, US

(73) Assignee: NANOBIODESIGN LIMITED, London SW7 2AZ (GB)

(21) Appl. No.: 10/537,612

(22) PCT Filed: Dec. 3, 2003
(86) PCT No.: PCT/GB03/05256

§ 371 Date: Jan. 26, 2006

(30) Foreign Application Priority Data
Dec. 4, 2002 (GB) 0228301.8

(51) Int. Cl. C12Q 1/26; C07H 21/04; C12P 21/06; C12N 9/02

(52) U.S. Cl. 435/25; 435/69.1; 435/189; 435/320.1; 435/325; 536/23.2

(57) ABSTRACT

The protein of this invention comprises a 4-α-helix bundle motif formed from the α-helices of rop (repressor of primer) and a redox centre. In this invention the redox centre is preferably haem and the iron is preferably coordinated to the α-helices of the rop structure via histidine residues. Such a protein is very stable and has the same specific activities as natural redox proteins with the increased stability in the interaction with electrode surfaces. Also provided by this invention is a method of engineering proteins to develop redox proteins from structures which previously had no redox functions.

This invention relates to engineering proteins so that they have the required properties to carry out a particular function.

A very attractive goal for chemistry and biology is the creation of engineered protein structures capable of the same specific activities as their natural counterpart, but without the complexity associated with cellular functions. This has been addressed either by the de novo synthesis of peptides using specified templates, or by mutating wild type redox proteins or more recently by modular assembly of existing proteins.

Protein engineering work has been carried out on many proteins including the E. coli repressor of primer (rop) a regulatory protein which binds nucleic acid and has a 4-α-helix bundle motif.

The 4-α-helix bundle is a commonly occurring, stable, structural motif. The 4-α-helix bundle is typified by an association of 4-α-helices that cross at an angle of about 20°. The helices may be linked by loops which can vary in their length, sequence and connectivity. The basic 4-helix bundle framework can support a diverse variety of activities, from oxygen transport in myohemerythrin to receptor binding in haematopoietic growth factors and RNA binding in rop.

Wild type rop is dimeric, each monomer being formed by two helices connected by a sharp bend. Predki and Regan (1995) describe producing a single chain rop. Their method separates helix 1 and 2 sequences of one rop molecule and places helix 1 before helix 1′ and places helix 2 after helix 2′ of a second rop (consisting of helices 1′ and 2′). The monomeric rop contains 4 α-helices in the order 1-1′-2′-2 and all a helices are expressed as a single polypeptide chain. This method of ordering the 4 α helices maintains the wild type N- and C-termini and the connection between helix 1′ and 2′.

This invention provides a protein comprising:

(a) a 4-α-helix bundle motif formed from the α-helices of rop (repressor of primer) and

(b) a redox centre.

The inventors have provided a stable protein capable of carrying out redox activities and other properties of the protein can be controlled.

This invention takes an existing simple and stable protein fold as a structure in which to insert new functions. This approach mimics the natural process of protein evolution, where within a protein superfamily with a particular structure a diversity of function can be found. The protein of this invention is based on the well-studied and understood 4-helix-bundle protein fold.

The protein of the current invention comprises a redox centre. A redox centre is an atom, group of atoms or moiety capable of accepting and donating electrons. The redox centre may be a metal atom such as iron or copper which is stable in two different oxidation states. The redox centre may be an iron sulphur centre which generally comprise two or four iron atoms bound to an equal number of sulphur atoms and to cysteine side chains. The redox centre may also be a haem group which comprises an iron atom held in the middle of a porphyrin structure. Alternatively the redox centre may not contain a metal and may be an organic redox centre such as FMN or FAD.

The redox centre may be coordinated to the protein structure formed from the α-helices of rop. This may be the case when the redox centre is metal such as an iron or copper atom. Other redox centres such as haem are associated to the α-helices of the protein scaffold. Residues such as histidine, leucine, methionine and cysteine of the α-helices in the protein structure can be used to co-ordinate the iron atom of the haem as axial ligands when the redox centre is haem.

Residues suitable to coordinate to the redox centre are introduced into the helices of rop, and any residues present in the wild type sequence that could cause bonding to occur at an inappropriate place are removed, by mutation of the polypeptide sequence.

Simple natural cytochromes have reported mid-point potentials covering the range of −475 to +310 mV. The redox midpoint potential of the protein of this invention lies in the range −485 to +320 mV. The midpoint potential can be altered depending on the use of the redox protein. A major determinant of the redox potential of haem proteins is thought to be haem exposure. In addition other factors have also been implicated including the polarity of the haem environment, the strength of the axial ligand field, and the electrostatic interactions between haem and its propionates. (Stellwagen (1978; Kassner, R. J. (1973); Valentino J. S. et al (1979) & Moore, G. R. (1983)). It has also been demonstrated that the redox potential of cytochrome b₅₆₂ can be varied by over 100 mV by changing the amino acids in the vicinity of the haem, (Springs, S. L. et al (2000));

A second aspect of this invention provides a method of producing the protein of this invention. Random mutagenesis or site directed mutagenesis is performed to engineer the protein such that it contains the necessary residues to enable redox centre binding to the protein. Therefore the DNA sequence which encodes the protein of this invention, either isolated or incorporated into a vector is also part of the invention. This sequence is then expressed in and the polypeptide purified from a microorganism. (Alternatively it is possible that the polypeptide can be produced using a solid phase peptide synthesis). The resulting polypeptide, which is capable of binding a redox centre, is then incubated with an excess of that redox centre to ensure incorporation into the protein.

A third aspect of this invention involves the use of the engineered protein. The protein of the current invention may be used as an electron carrier either on its own or in a series of electron carriers. A series of electron carriers may be an electron transport chain, or it may include an electrode. An electron transfer chain transports electrons from a higher to a lower energy level along a series of electron carrier molecules. An electron carrier molecule is a molecule that transfers an electron from a donor molecule to an acceptor molecule. An electron acceptor is a molecule that takes up electrons easily, thereby gaining an electron and becoming reduced, whereas an electron donor is a molecule that easily gives up an electron, becoming oxidised in the process. Therefore the protein of this invention can be used in a method which involves passing electrons along a sequence of electron carriers, in which each electron carrier is reduced and then oxidised (or vice versa) by electron movement and the sequence of electron carriers includes the protein of this invention. The electron transport chain may comprise natural or synthetic electron carriers. The protein of this invention can be used in a method involving passing electrons along a sequence of electron carriers, in which each electron carrier is reduced and then oxidised or vice versa by electron movement and the protein of this invention forms part of the sequence of electron carriers. In such a method electrons are generally moved along a gradient of electron carriers with successively lower or higher redox potential.

An alternative use of the protein of this invention is as part of an apparatus comprising the protein associated with an electrode in a manner that electrons may be passed from one to the next. The protein may be bound or adsorbed onto the electrode. This includes use of the protein of this invention in cyclic voltammetry, which is used to provide information about the redox protein's mid-point potential.

As has been mentioned above the 4-α-helix bundle of rop protein forms a stable structure. The redox protein of the present invention is also stable and has an unfolding free energy when denaturant is added to the protein of ΔG_{obs}^{H}₂^{O}≧y wherein y≧3.0 kcal/mol. The redox protein of this invention contains mutations which allow bonding of the redox centre. In addition to incorporation of a residue or residues for bonding to the redox centre a aromatic amino acids such as tryptophan can be inserted into the protein. Insertion into the protein structure of aromatic amino acids provides a method of spectrophotometric detection of the protein. It is necessary to ensure that the insertion of residues allowing spectrophotometric detection does not destabilise the protein.

### BRIEF DESCRIPTION OF THE FIGURES

FIG. 1 shows ribbon diagram representations of rop. The 3D models for monomeric rop were derived from the wild type dimeric rop model using the BIOPOLYMER and DISCOVERS modules within Insight II.

FIG. 2 shows the absorbance spectra of a bis-Histidine haem binding rop. The data were collected on a Hewlett-Packard 8453 UV-visible photodiode array spectrophotometer.

FIG. 3 shows the electrochemical properties of the rop haem protein.

FIG. 4 shows the DNA and protein sequence of wild type dimeric rop with the α-helix regions shown in boxes. Also shown are psp 7 amplification upstream sequence and asp4 amplification downstream sequence.

FIG. 5 shows the DNA and protein sequence of monomeric rop (S55) (created by Predki & Regan) with the α-helix regions highlighted. Also shown are psp 7 amplification upstream sequence and asp4 amplification downstream sequence.

FIG. 6 shows a protein sequence of a redox protein of the present invention formed from the α-helices of rop, produced according to example 1.

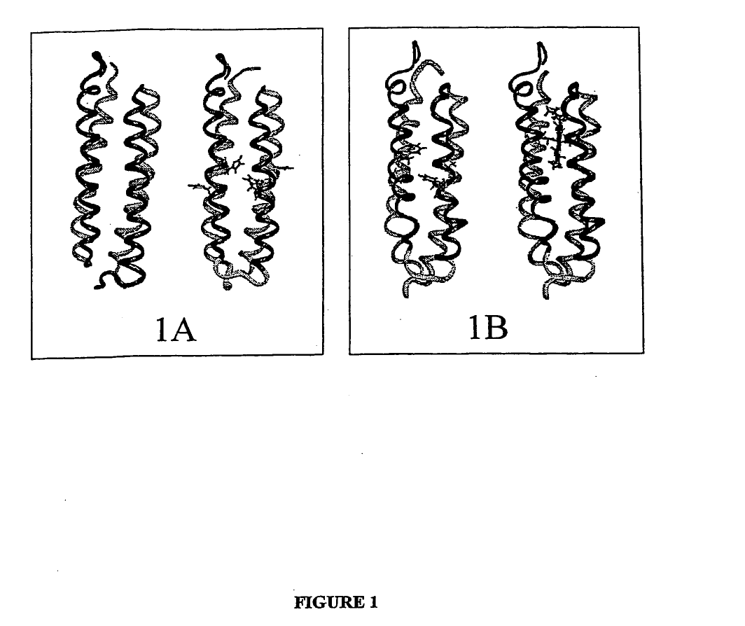

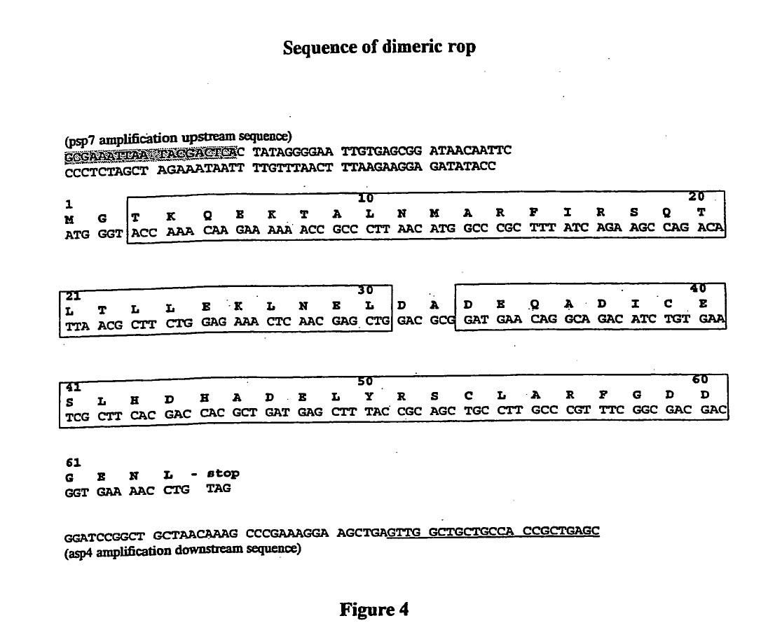

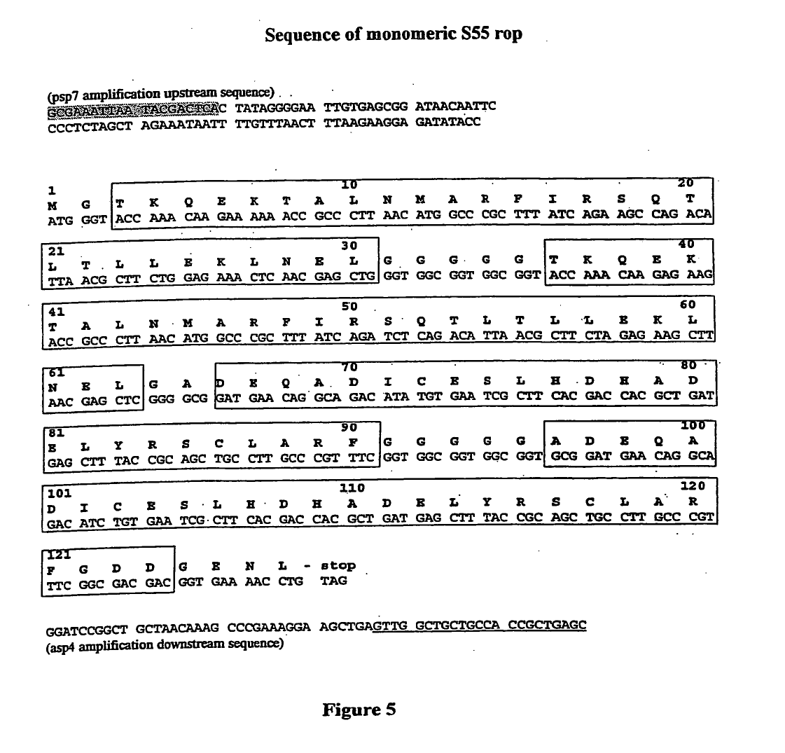

In the preferred type of protein of this invention the 4α-helix bundle structure used to develop the proteins of this invention is monomeric rop as described above. This protein is developed from the wild type dimeric rop which is shown in FIG. 4 and sequence ID Nos 5 and 6. In FIG. 4 the portions of the sequences enclosed in boxes are the α-helices. In the momomeric protein all 4 helices are expressed as a single polypeptide chain. Monomeric rop is shown in FIG. 5 and sequence ID Nos 7 and 8. In FIG. 5 the portions of the sequence enclosed in boxes are the α-helices. When specific residues are referred to the numbering system relates to the monomeric rop developed previously and shown in FIG. 5. A structure for monomeric rop was generated by homology modeling using the program Insight II (MSI, San Diego) using the NMR structure as a template. This is shown in FIG. 1 which shows the design of monomeric rop. In this embodiment native dimeric rop (left) was engineered into a single polypeptide chain monomeric variant (right) facilitated by the introduction of two 5×Gly loops (Predki & Regan, 1995). The native His residues (76, 78, 107, 109) are shown in stick representation. In the protein of the present invention these His residues are removed as described below and residues necessary for interaction with the redox centre are introduced into the polypeptide. Removed means a particular amino acid is no longer at that position. Preferably this is achieved via substitution, however deletion may be a possibility. Introduced means a particular amino acid is now found at that position. Preferably this is achieved via subtitution, however insertion may also be a possibility. Monomeric rop provides a better template for engineering than its dimeric precursor because of the ability to introduce single mutations.

As can be seen from FIG. 5 the first two helices and the second two helices of monomeric rop have the same sequence. The helix secondary structure may start one residue earlier in helix 4 (with A) than helix 3 because the GGGGG loop preceding it is long enough to allow reorientation, whilst the α-helix secondary structure in helix 3 probably begins at D as only one G is present between helices 2 and 3, therefore A may still be part of the loop region.

In the mutated monomeric rop proteins of this invention the α-helix regions are generally conserved and preferably the helices of the 4α-helix bundle of the protein of this invention show 60% or 70%, preferably 80 similarity or identity with the α-helix regions indicated in FIGS. 4 and 5 and shown in sequence ID No. 1, 2, 3 and 4. The variation is generally introduced by removing native His residues 76, 78, 107 and 109 as they could lead to inappropriate binding of a redox centre, or by including residues required to incorporate the redox centre into the protein or by altering residues in the vicinity of the redox centre. More variation may be introduced by residues which allow detection of the protein, for example including aromatic amino acids such as tryptophan. The loops between each α-helix may, however, vary significantly (from that shown in FIG. 5 and sequence ID Nos 7 and 8) in terms of their length, sequence and connectivity, provided that the loop regions allow the α-helices to come together in 3D space to form the 4α-helix bundle.

The terms as used above, “similarity” and “identity” are known in the art. The use of the term “identity” refers to a sequence comparison based on identical matches between correspondingly indentical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison Wis.

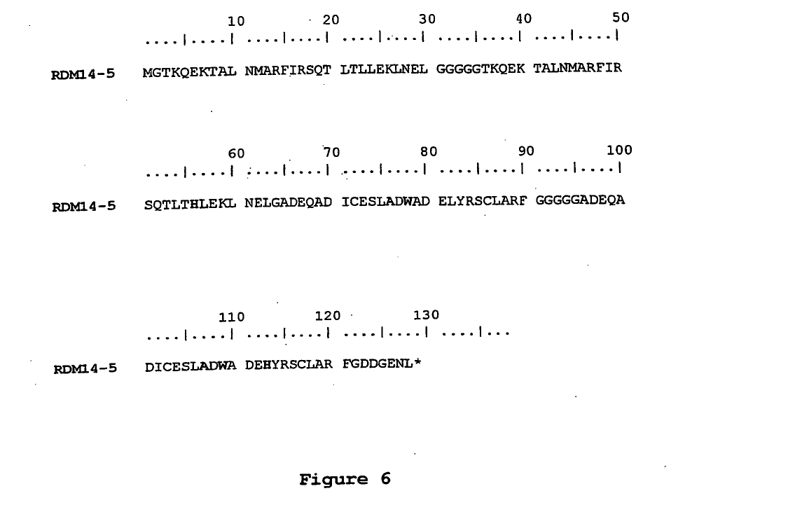

A more preferred protein of this invention is based on monomeric rop as described above and contains the following mutations, the histidines at H76H78 H107 and H109 are substituted for other residues. In one embodiment H76A and H107A substitutions create a binding site for a haem group. In addition the mutations H78W and H109W may occur to create a means of spectrophotometric detection of the novel redox protein. Further mutations may also be incorporated into this protein for example L56H and L113H. The sequence of the especially preferred redox protein with mutations H76A, H78W, H107A, H109W and L56H and L113H is given in sequence ID No. 11 and in FIG. 6.

All the proteins of this invention are stable and have an unfolding free energy as described above. The stability of the proteins of this invention is comparable with or greater than the stability of wild type rop itself.

### EXAMPLE 1

### Production of Monomeric rop Possessing a haem Group

The monomeric rop constructs, (S55), was the starting point for the rational design strategy (Munson et al 1994; P F Predki & Regan, 1995). Site directed mutagenesis was performed either by the “Megaprimer method” or using a method based on the “Quik Change” method (Stratagene Europe) (Trade mark) in which a plasmid is amplified using two complementary oligonucleotides containing the desired mutation. DNA manipulations were carried out in E.coli DH5 α or E-coli XL1-Blue (stratagene Europe). When the “Megaprimer method” was used, the rop gene was amplified by PCR using flanking primers psp7 and asp4 (sequence ID No 9 and 10 respectively) and mutations were confirmed by sequencing of the PCR product. Oligonucleotides were obtained from Amersham Pharmacia Biotech (Table 1).

TABLE 1
Oligonuleotides used to engineer the haem binding mutants.

| | | |
|---|---|---|
| Name | Oligonucleotide Forward | Oligonucleotide Reverse |

| | | |
|---|---|---|
| psp7 | gcggaaattaatacgactca | |
| asp4 | | gctcagcggtggcagcagccaac |
| H76A, H78A | gaatcgcttgccgacgccgctgatg | |
| H76A, H78W | gaatcgcttgctgactgggctgat | |
| H107A, H109W | ctcagacattaacgcacctagagaagcttaacg | gagctcgttaagcttctctaggtgcgttaatgtctgag |
| L56H | agctc | |
| L113H | cgccgctgatgagcattaccgcagctgcc | ggcagctgcggtaatgctcatcagcggcg |
| L63M | gaagcttaacgagatgggggcggatgaacagg | cctgttcatccgcccccatctcgttaagcttc |
| F121H | agctgccttgcccgtcacggcgacgacggtg | caccgtcgtcgccgtgacgggcaaggcagct |

The initial step was to remove potential non-specific ligands by the replacement of endogenous histidine residues creating the mutant H76A, H78W, H107A, H109W, see FIG. 1A. A haem binding site was created in this protein by the further mutations L56H, L113H, see FIG. 1B. FIG. 1B shows the design of haem binding sites in monomeric rop. A monomeric rop variant (left) lacking the native His residues (H76A, H78W, H107A, H109W) was produced by site directed mutagenesis. Mutations L56H, L113H were incorporated to introduce haem ligands. A sequential approach was taken to incorporate multiple mutations into rop. Constructs leading to the creation of a haem-binding-rop variant are listed in Table 2 below. The RDM14.5 construct, containing the (L56H, L113H) haem binding site (rop-56H/113H) was made by the subcloning of the appropriate fragments from RDM10-b4 and QC11-J1.

TABLE 2
Rop constructs produced for this study with mutations from the
S55 sequence.

| Construct | Mutations |
|---|---|
| S55 | |
| JW1 | H107A, H109A |
| JW3sub | H76A, H78A, H107A, H109A |
| JW2 | H76A, H78W, H107A, H109W |
| JW7 | H76A, H78W, H107A, H109A |
| RDM2.1 | H76A, H78W, H107A, H109W, L63H |
| QC3.5 | H76A, H78W, H107A, H109W, L63H, F121H |
| QC7.2 | H76A, H78W, H107A, H109W, L63M, F121H |
| RDM10b4 | H76A, H78W, H107A, H109W, L56H, G64R |
| QC11-J1 | H76A, H78W, H107A, H109W, L113H |
| RDM14.5 | H76A, H78W, H107A, H109W, L56H, L113H |

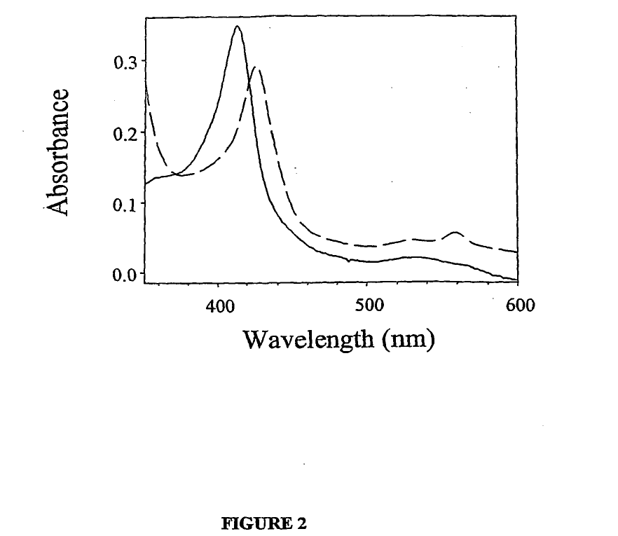

The haem binding variant was heterologously expressed from the pMR103 plasmid in E. coli BL21(λDE3) and purified to homogeneity by standard techniques. Haem was incorporated into the apo-protein by addition of a 4-fold molar excess of hemin chloride solubilised in DMF, incubation overnight, and removal of free haem by iron exchange chromatography (DEAE sepharose FF, Amersham Pharmacia Biotech UK Ltd). The resulting haem binding rop protein has spectral properties characteristic of native b-type cytochromes. FIG. 2 shows spectra of bis-Histidine haem bind rop variant in oxidized and reduced form. Solid line: 20 μM haem binding monomeric rop variant L56H, L113H in 10 mM sodium phosphate buffer, pH80. Dashed line: 20 μM rop following reduction by sodium dithionite. The oxidized protein has a λₘₐₓ at 413 nm, which upon reduction with sodium dithionite shifts to 426 nm with distinct α and β peaks at 559 nm and 531 nm respectively. The spectral characteristics are typical of b-type cytochromes with a six coordinate low spin iron, and this is a strong indication that the haem group has been incorporated into the helix bundle as predicted in the design.

### EXAMPLE 2

### Testing the Redox Properties of this Novel haem Protein

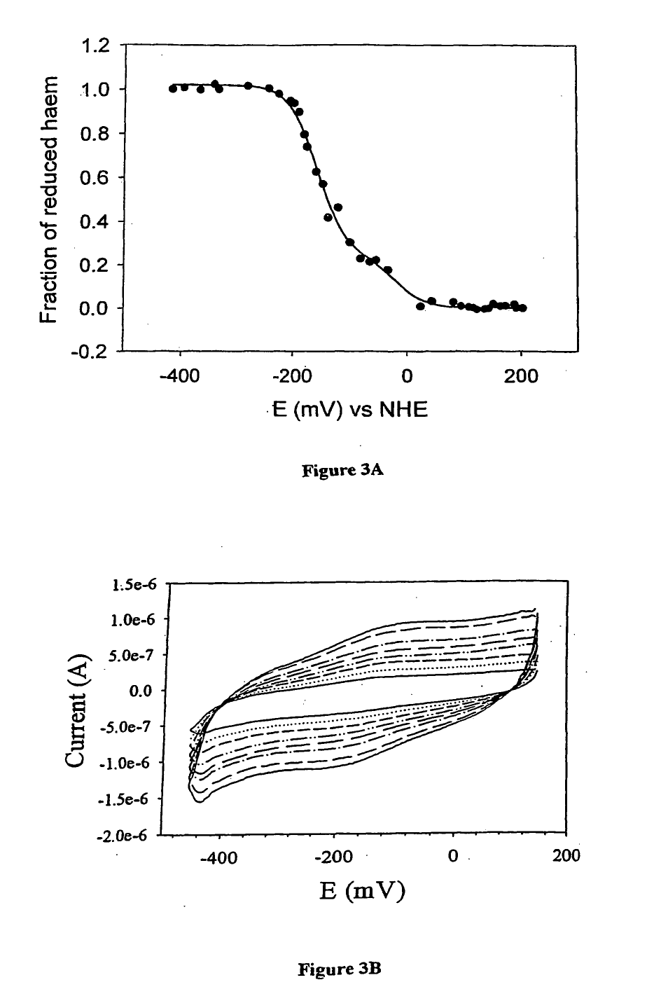

The redox properties of this novel haem protein rop-56H/113H were determined by spectroelectrochemistry and cyclic voltammetry and are presented in FIG. 3. FIG. 3A shows redox titration of rop variant with bound haem. Titration of sodium dithionite into protein solution (40 μM) was performed anaerobically. Following equilibration the UV-visible spectrum was obtained (Hewlett-Packard 8453 spectrophotometer) and the potential measured simultaneously (Autolab PGSTAT10 potentiostat). Titrations were performed at 20° C. in pH 7.8 buffer.

FIG. 3B shows cyclic voltammograms of rop variant with bound haem. Cyclic voltammetry was performed on a bare glassy carbon electrode over a range of scan rates.

Spectrophotometic titrations were performed in an anaerobic a quartz cuvette (Hellma (England) Limited UK) equipped with a platinum-mesh working and an Ag/AgCl reference electrode, according to the procedure described in Dutton, P. L., 1978. Ambient redox potentials were adjusted by addition of aliquots (<5 μl) of sodium dithionite or potassium ferricyanide. Titrations were performed in 50 mM potassium phoshate, pH 7.5. Electrode-solution mediation was facilitated by the following mediators: phenazine methosulphate (5 μM), duraquinone (5 μM), 2-hydroxy 1,4 napthoquinone (5 μM) benzyl viologen (2 μM), indigocarmine (0.5 μM), resorufin (0.5 μM). The modified cuvette was flushed with oxygen-free nitrogen before and during the measurements. After each addition of sodium dithionite (0.5 μl of a 10 mM solution) the protein was allowed to equilibrate and at each potential, the optical spectrum was recorded using a Hewlett-Packard 8452 diode array spectrophotometer. The fraction of the reduced protein was estimated at each potential by following the shift in Soret peak (413 to 426 nm) and was plotted against potential. The data points were fitted to the Nernst equation with n=1:
E=Em+(RT/nF) In ([ox]/[red])
where [ox] and [red] are the concentration of oxidised and reduced haem respectively, E is the solution redox potential at equilibrium, n is the number of electrons, Em is the mid-point potential, F is the Faraday constant, R is the gas constant and T is the absolute temperature.

The redox titrations with sodium dithionite (FIG. 3A) show one larger component (78%) at −154±2 mV and one smaller (22%) at 17±9 mV, relative to the normal hydrogen electrode. The presence of two reduction potentials is likely to be due two non-equivalent orientations in the engineered rop. This is at present being investigated. Only one mid point potential of Em=−134±13 mV could be measured using cyclic voltammetry on a bare glassy-carbon electrode using a modified Hagen cell (FIG. 3B).

These reduction potentials are consistent with those measured for natural cytochromes and heme-containing synthetic peptides, that cover the range of −475 to +310 mV. The properties of the rop heme binding protein are at present being optimised with the creation of a cavity that accommodates the heme macrocycle with a unique orientation, with the possibility of tuning the redox potential. It has been demonstrated that the redox potential of cytochrome b562 can be varied by over 100 mV by changing the amino acids in the vicinity of the heme and Dutton and coworkers demonstrated that by utilizing alternative porphyrins it was possible to modulate the redox potential of their designed heme protein by as much as 225 mV. It is anticipated that the redox properties of this designed protein could likewise be “tuned” as required for particular applications.

### EXAMPLE 3

### Stability of rop

The protein of this invention shows a high degree of stability which may be measured as the unfolding free energy when a denaturant is added to the protein, of ΔG_{obs}^{H}₂^{O} is greater than or equal to y wherein y≧3.0 kcal/mol.

The stability test refers to the chemical stability. This was measured in solution by detecting changes of the circular dichroism signal at 222 nm and changes of fluorescence anisotropy signal of the protein upon additions of guanidinium hydrochloride. Experiments were carried out by addition to the protein of denaturant (guanidinium hydrochloride) to reach final concentrations from zero to 6.5 M. The parameter that is related to the stability is the ΔG of unfolding in water (ΔG_{obs}^{H}₂^{O}).

The data derived from the CD and fluorescence anisotropy were analysed assuming a two state model, f_{F}+f_{U}=1, where f_{F} and f_{U} represent the fraction of total protein in the folded and unfolded state respectively. The equilibrium constant K_{U} and the free-energy change ΔG_{obs}^{U} for the unfolding reaction were calculated from:
K_{U}=f_{U}/(1−f_{U})=f_{U}/f_{F}
G_{obs}^{U}=−RT In K_{U}
According to the linear extrapolation method, the unfolding free energy depend linearly from the denaturant concentration (Shellman, J. A., 1987):
G_{obs}^{U}=G_{obs}^{H}₂^{O}−m*[GuHCl]

The results are shown in table 3.

TABLE 3
Thermodynamic parameters characterizing the unfolding process

| | DG₁ | m₁ | DG₂ | m₂ |
|---|---|---|---|---|
| Sample | (kcal/mol) | (kcal/mol M) | (kcal/mol) | (kcal/mol M) |
| S55 | 8.7 ± 0.5 | 2.4 ± 0.3 | | |
| JW2 | 8.7 ± 0.8 | 1.9 ± 0.5 | | |
| QC7.2 | 3.1 ± 0.5 | 2.9 ± 0.2 | 3.9 ± 0.5 | 1.1 ± 0.3 |
| RDM14.5 | 3.5 ± 0.6 | 3.0 ± 0.9 | 4.0 ± 0.5 | 1.0 ± 0.2 |

The mutant Jw2, the parent from which all of the mutants containing the haem have been obtained, shows a ΔG of unfolding of 8.7 kcal/mol, as also shown for the original monomeric rop S55, 8.7 kcal/mol.

A two-step transition model was necessary to adequately fit the data of RDM14.5 and QC7.2, resulting in a total free energy change of 7.5±1.1 kcal/mol and 7.0±1.0 kcal/mol respectively (see table 3). The unfolding of the starting construct proteins S55 and JW2 have been also reported for comparison in table. In these cases the unfolding process was satisfactorily represented by a simple two-state transition with similar free energy changes (table 3). These findings suggested that the haem bound proteins undergo a different unfolding mechanism due to the presence of the haem group. This hypothesis has been tested measuring the changes of QC7.2 and RDM14.5 absorption spectrum in the visible region, as a function of guanidinium hydrochloride (GdHCl) concentration. Most of the signal at 415 nm is already lost at 3 M GdHCl, confirming the loosening of the haem group.

### REFERENCES

Dutton, P. L. (1978) in Methods in Enzymology LIV pp 411-435, ed Fleisher, S & Packer, L., Academic Press, New York.
Hagen, W. R. (1989) Eur J. Biochem 182 523
Heering H. A. & Hagen W. R. (1996) J. Electroanal Chem 404, 249
Kassner, R. J. (1973) J. Am. Chem. Soc., 95, 2674
Moore, G. R., (1983) FEBS Lett, 161, 171
Munson, M., Obrien, R., Sturtevant, J. M. & Regan, L. (1994) Protein Sci, 3, 2015-2022
Munson, M., Predki, P. F. & Regan, L. (1994) Gene 144, 59-62
Predki, P. F. & Regan, L. (1995) Biochemistry 34, 9834-9839
Shellman, J. A. (1987) Biopolymers 26, 549-559
Springs, S. L., Bass, S. E. & McLendon, G. L. (2000) Biochemistry, 39, 6075-6082
Stellwagen, E. (1978) Nature 275, 73
Valentine, J. S. Sheridan, R. P., Allen, L. C. & Kahn, P. C. (1979) Proc. Notl. Acad. Sci USA, 76, 1009
Wilson, J. R., Caruana, D. J. & Gilardi, G. Chem. Com. (in print)

1. A protein comprising:
a) 4-α-helix bundle motif formed from the α-helices of ROP (repressor of primer) and
b) a redox centre.

2. The protein of claim 11 wherein the redox centre comprises a metal atom which is stable in two different oxidation states.

3. The protein of claim 1, wherein the redox centre is bound to the protein, by coordination by one or more of histidine, leucine, methionine or cysteine residues.

4. The protein of claim 1, wherein the redox centre is covalently bound to the protein.

5. The protein of claim 1 which has a redox mid-point potential in the range of −485 to +320 mV.

6. The protein of claim 1 which has α-helix regions each having at least 60% similarity or identity with the α-helix regions of sequence ID Nos. 1 and 3.

7. The protein of claim 6, wherein said four α-helix regions are connected by loops.

8. The protein of claim 7, wherein the four α-helices are joined in the order 1-1′-2′-2.

9. The protein of claim 1 which is formed by connecting two wild type ROP proteins to obtain the 4-helix bundle as one continuous polypeptide having at least 60% similarity or identity with sequence ID No. 8.

10. The protein of claim 9, wherein the histidine residues corresponding to H76, H78, H107 and H109 in sequence ID No. 8 are removed.

11. The protein of claim 9, wherein histidine, leucine, methionine or cystein residues are introduced one or both positions corresponding to 56 and 113 in sequence ID No. 8.

12. The protein of claim 1 which has a haem redox centre coordinated to the 4-α-helix bundle motif via two histidine residues.

13. The protein of claim 12 which has a mid-point potential in the range −400 mV to +300 MV.

14. The protein of claim 12 which has the sequence as indicated by sequence ID No. 11.

15. The protein of claim 1 which has a stability, measured as the unfolding free energy when denaturant is added to the protein, of ΔG_{obs}H₂O≧ wherein y≧3.0 kcal/mol.

16. A method of producing the protein of claim 1 comprising
i) expressing all four α-helices as a single polypeptide chain;
ii) engineering the required mutations to enable redox centre binding;
iii) expressing and purifying, or producing the redox centre binding mutant; and
iv) incubating the mutant with an excess of the redox centre to produce the protein.

17. A nucleotide sequence which encodes the protein of claim 1 or a fragment thereof.

18. A vector comprising the nucleotide sequence of claim 17.

19. (canceled)

20. A method of passing electrons along a sequence of electron carriers, in which each electron carrier is reduced and then oxidized or vice versa by electron movement and the sequence of electron carriers includes the protein of claim 1.

21. An apparatus comprising the protein of claim 1 associated with an electrode.

22. An apparatus according to claim 21 wherein the protein is absorbed onto an electrode.

23. A protein according to claim 2 in which the redox centre is an iron sulfur centre.

24. A protein according to claim 1 in which the redox centre does not contain a metal atom.

25. A protein according to claim 24 in which the redox centre is FMN or FAD.

26. A protein according to claim 6 in which the α-helix regions each have at least 80% similarity or identity with the α-helix regions of sequence ID No. 1.

27. A protein according to claim 9 in which the continuous polypeptide has at least 80% similarity or identity with sequence ID No. 8.
